(19) United States
(12) Patent Application Publication
(10) Pub. No.: US 2020/0000626 A1
(43) Pub. Date: Jan. 2, 2020

(54) Sleep Aid Devices and Related Methods

(71) Applicant: AZ TMJ, PLLC, Scottsdale, AZ (US)

(72) Inventors: Stan Farrell, Scottsdale, AZ (US); Brandon Miller, Scottsdale, AZ (US); Ahmad Albaba, Gilbert, AZ (US)

(21) Appl. No.: 16/439,607

(22) Filed: Jun. 12, 2019

Related U.S. Application Data
(60) Provisional application No. 62/684,550, filed on Jun. 13, 2018.

(51) Int. Cl. A61F 5/56

(52) U.S. Cl. CPC A61F 5/566

(57) ABSTRACT

Implementations of sleep aid devices may include an upper portion configured to receive a user's upper teeth, the upper portion including a first upper projection and a second upper projection. Implementations may include a lower portion configured to receive a user's lower teeth coupled to the upper portion, the lower portion including a first lower projection and a second lower projection. A rear surface of the first lower projection may be configured to contact a front surface of the first upper projection. A rear surface of the second lower projection may be configured to contact a front surface of the second upper projection. The sleep aid device may be configured to one of shift forward the user's mandible or increase the user's occlusal clearance.

### CROSS REFERENCE TO RELATED APPLICATIONS

This document claims the benefit of the filing date of U.S. Provisional Patent Application 62/684,550, entitled “Sleep Aid Devices and Related Methods” to Farrell which was filed on Jun. 13, 2018, the disclosure of which is hereby incorporated entirely herein by reference.

### BACKGROUND

### 1. Technical Field

Aspects of this document relate generally to sleep aid devices. More specific implementations involve sleep aid devices used to treat sleep apnea.

### 2. Background

Sleep aid devices have been used to treat sleep disorders, such as obstructive sleep apnea (OSA). Sleep aid devices may include respiratory ventilation. An example of a sleep aid device is a continuous positive airway pressure (CPAP) machine which may use air pressure to keep the airway clear of obstructions.

### SUMMARY

Implementations of sleep aid devices may include an upper portion configured to receive a user's upper teeth, the upper portion including a first upper projection and a second upper projection. Implementations may include a lower portion configured to receive a user's lower teeth coupled to the upper portion, the lower portion including a first lower projection and a second lower projection. A rear surface of the first lower projection may be configured to contact a front surface of the first upper projection. A rear surface of the second lower projection may be configured to contact a front surface of the second upper projection. The sleep aid device may be configured to one of shift forward the user's mandible or increase the user's occlusal clearance.

Implementations of a sleep aid device may include one, all, or any of the following:

A bottom surface of the first upper projection and the second upper projection may be configured to contact the lower portion.

The sleep aid device may be configured to shift forward the user's mandible and increase the user's occlusal clearance.

The upper portion and the lower portion may be configured to form an opening at the front of the device when the lower portion is coupled to the upper portion.

The lower portion may include a plurality of slots and the upper portion may include a plurality of slots. Each of the plurality of slots may be configured to receive a plurality of elastics.

The device may be configured to prevent a user's soft tissue from blocking an airway of the user.

Implementations of sleep aid devices may include an upper portion including an upper shell configured receive to a user's upper teeth, an upper bar coupled below the upper shell, and a first upper projection and a second upper projection coupled below the upper shell. The first upper projection and the second upper projection may extend from a rear portion of the upper portion. Implementations may include a lower portion including a lower shell configured to receive a user's lower teeth, a lower bar coupled over the lower shell, and a first lower projection and a second lower projection coupled over the lower shell. A rear surface of first lower projection may be configured to contact a front surface of a first upper projection and a rear surface of the second upper projection may be configured to contact a front surface of the second upper projection. The first lower projection and the second lower projection may be configured to shift forward a user's mandible. The first lower projection and the second lower projection may be configured to provide occlusal clearance for a user.

Implementations of a sleep aid device may include one, all, or any of the following:

An entire bottom surface of the first upper projection and an entire bottom surface of the second upper projection may be configured to contact the lower bar.

An entire upper surface of the first lower projection and an entire upper surface of the second lower projection may be configured to contact the upper bar.

The upper shell and the lower shell may be formed to match the user's teeth.

The front surface of the first upper projection may slope towards the rear of the device from the upper shell.

The upper bar and the lower bar may be configured to provide occlusal clearance for the user.

A height of the first lower projection may be greater than a height of the first upper projection.

The upper shell may include a recess formed in a front sidewall of the upper shell.

The lower shell may include a recess formed in a front sidewall of the lower shell.

Implementations of a sleep aid device kit may include an upper portion configured to receive a user's upper teeth, the upper portion including a first upper projection and a second upper projection formed at a rear portion of the device. Implementations may include a minimum lower portion configured to receive the user's lower teeth, the minimum lower portion including a first minimum projection and a second minimum projection. Implementations may include a maximum lower portion configured to receive the user's lower teeth, the maximum lower portion including a first maximum projection and a second maximum projection. A rear surface of the first minimum projection and the first maximum projection may be configured to contact a front surface of the first upper projection. A rear surface of the second minimum projection and the second minimum projection may be configured to contact a front surface of the second upper projection. One of a height of the projections of the maximum lower portion may be greater than a height of the projections of the minimum lower portion or a length of the projections of the maximum lower portion may be greater than a length of the projections of the minimum lower portion.

Implementations of a sleep aid device kit may include one, all, or any of the following:

Implementations may include a first intermediate lower portion including a first intermediate projection and a second intermediate projection. A height of the first intermediate projection may be greater than the height of the first minimum projection and less than the height of the first maximum projection.

Implementations may include a second intermediate lower portion.

Each lower portion may configured to shift a user's mandible forward and to increase a user's occlusal clearance.

Implementations may include a second upper portion configured to receive a user's upper teeth.

The foregoing and other aspects, features, and advantages will be apparent to those artisans of ordinary skill in the art from the DESCRIPTION and DRAWINGS, and from the CLAIMS.

### BRIEF DESCRIPTION OF THE DRAWINGS

Implementations will hereinafter be described in conjunction with the appended drawings, where like designations denote like elements, and:

FIG. 1 is a side view of the sleep aid device;

FIG. 2 is a top perspective view of a lower portion of a sleep aid device;

FIG. 3 is a is a side view of a lower portion of a sleep aid device;

FIG. 4 is a front view of a lower portion of a sleep aid device;

FIG. 5 is a rear view of a lower portion of a sleep aid device;

FIG. 6 is a bottom view of a lower portion of a sleep aid device;

FIG. 7 is a top view of a lower portion of a sleep aid device;

FIG. 8 is a top perspective view of an upper portion of a sleep aid device;

FIG. 9 is a side view of an upper portion of a sleep aid device;

FIG. 10 is a front view of an upper portion of a sleep aid device;

FIG. 11 is a rear view of an upper portion of a sleep aid device;

FIG. 12 is a bottom view of an upper portion of a sleep aid device;

FIG. 13 is a top view of an upper portion of a sleep aid device;

FIG. 14 is a top perspective view of a second implementation of a lower portion of a sleep aid device;

FIG. 15 is a side view of a second implementation of a lower portion of a sleep aid device;

FIG. 16 is a top perspective view of a third implementation of a lower portion of a sleep aid device;

FIG. 17 is a side view of a third implementation of a lower portion of a sleep aid device;

FIG. 18 is a top perspective view of a fourth implementation of a lower portion of a sleep aid device;

FIG. 19 is a side view of a fourth implementation of a lower portion of a sleep aid device;

FIG. 20 is a bottom view of a fourth implementation of a lower portion of a sleep aid device;

FIG. 21 is a front view of a fourth implementation of a lower portion of a sleep aid device;

FIG. 22 is a top perspective view of a fourth implementation of a lower portion of a sleep aid device with a first implementation of an attachment;

FIG. 23 is a top perspective view of a fourth implementation of a lower portion of a sleep aid device with a second implementation of an attachment;

FIG. 24 is a top perspective view of a second implementation of an upper portion of a sleep aid device;

FIG. 25 is a side view of a second implementation of an upper portion of a sleep aid device;

FIG. 26 is a front view of a second implementation of an upper portion of a sleep aid device;

FIG. 27 is a top view of a second implementation of an upper portion of a sleep aid device;

FIG. 28 is a top perspective view of a second implementation of an upper portion of a sleep aid device with a first implementation of an attachment; and

FIG. 29 is a top perspective view of a second implementation of an upper portion of a sleep aid device with a second implementation of an attachment.

### DESCRIPTION

This disclosure, its aspects and implementations, are not limited to the specific components, assembly procedures or method elements disclosed herein. Many additional components, assembly procedures and/or method elements known in the art consistent with the intended sleep aid device will become apparent for use with particular implementations from this disclosure. Accordingly, for example, although particular implementations are disclosed, such implementations and implementing components may comprise any shape, size, style, type, model, version, measurement, concentration, material, quantity, method element, step, and/or the like as is known in the art for such sleep aid devices, and implementing components and methods, consistent with the intended operation and methods.

Particular implementations of sleep appliances are disclosed in this document. Specifically, lower jaw repositioning devices such as mandibular repositioning devices (MRDs) and mandibular advancement devices (MADs) are described in this application. The sleep aid devices disclosed herein may, by repositioning the lower jaw or mandible, prevent the tongue or other soft tissue of a user from blocking an individual's airway. The implementations disclosed herein may accomplish this through shifting the jaw forward and/or through increasing the occlusal clearance of a user. As used herein, “occlusal clearance” is define as the space between the teeth of a user's upper jaw and the teeth of the user's lower jaw. Further, as used herein, “front,” “rear,” “upper,” and “lower,” have the meaning of the respective front, rear, upper, and lower portion of the device according to the in-use orientation of the device when it is in a user's mouth. Accordingly, the “lower portion” of the device refers to the portion configured to couple over a user's lower teeth while the “upper portion” refers to the portion of the device configured to couple over the user's upper teeth. Similarly, the front of the device would be the end of the device near the user's front teeth and the rear of the device would be the portion of the device towards the back of the user's mouth. While the implementations disclosed herein are described in the context of a sleep aid device, the device and method disclosed herein may be used for other MRDs or MADs that may not be sleep aid devices.

Referring to FIG. 1, a side view of a sleep aid device is illustrated. As illustrated by FIG. 1, the sleep aid device 2 includes an upper portion 4 and a lower portion 6. The lower portion 6 is configured to fit over and receive a user's lower teeth, and the upper portion 4 is configured to fit over and receive a user's upper teeth. As illustrated, the upper portion 4 is configured to engage with the lower portion 6 to shift the lower portion a predetermined amount. Accordingly, by shifting the lower portion 6, a user's mandible may in turn be shifted, advanced, and/or repositioned through the movement of the lower portion when the upper portion 4 is coupled to and engages with the lower portion 6.

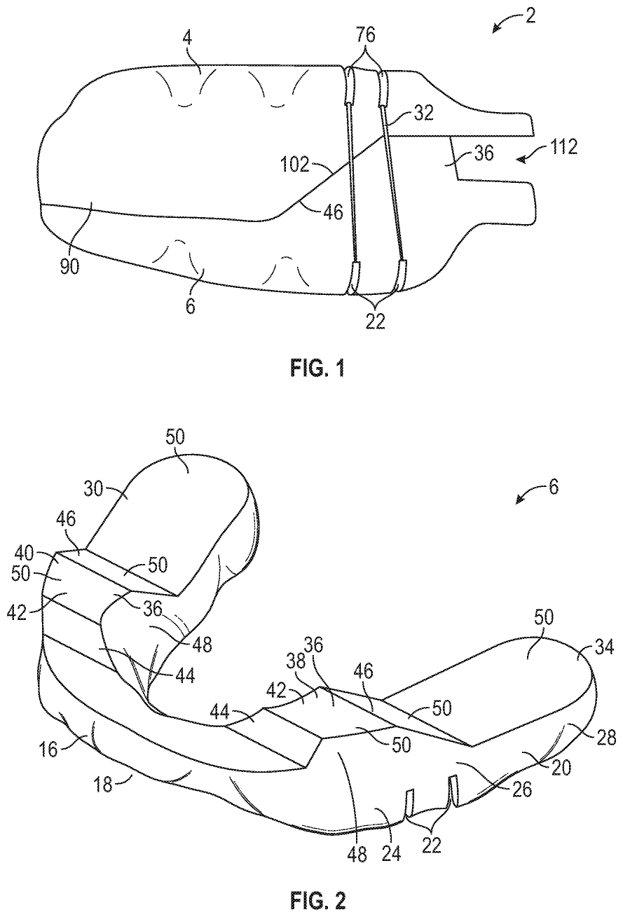

Referring to FIG. 2, a top perspective view of a lower portion of a sleep aid device is illustrated. In various implementations, and as illustrated, the lower portion 6 may be a single unitary piece without any kind of seam. In other implementations, the lower portion 6 may be made from various separable or separately formed portions which are combined together. In various implementations, the lower portion may be made from various materials, such as, by non-limiting example, plastics, polymers, or monomers. In particular implementations, the material may be biocompatible class II(a) material approved by the U.S. Food and Drug Administration (FDA) for long term use in the mouth. In particular implementations, the material may be a monomer based on acrylic esters. An example of such a material is the monomer marketed under the tradename NEXTDENT® Ortho Clear by 3D Systems, Inc., of Circle Rock Hill, S.C. In various implementations, the lower portion 6 may be a single material, while in other implementations differing materials may be included in the lower portion, whether in separately formed portions or through layering of different materials on the same structure.

Referring to FIG. 6, a bottom view of a lower portion of a sleep aid device is illustrated. In various implementations, the lower portion 6 may include a bottom tray 8. The bottom tray 8 may be a portion of the lower portion 6 molded to fit over an individual's lower teeth. In various implementations, the bottom tray 8 may be made to specifically match a single user's lower teeth. In various implementations, the bottom tray 8 may include, by non-limiting example, a crown matching surface 10 configured to match the crowns of an individual's lower teeth, a lingual matching surface 12 configured to match the lingual structure of an individual's lower teeth, a facial matching surface 14 configured to match the facial structure of an individual's lower teeth, and any combination thereof. As used herein, lingual structure refers to the inner surface of a user's teeth, or surface facing the tongue, and facial structure refers to the outer surface of a user's teeth, or surface nearest the face. Referring back to FIG. 2, in various implementations the front 16 of the lower portion 6 may include a frontal space 18 where the bottom tray 8 does not include a corresponding facial matching surface 14 over the entirety of the front teeth. In various implementations, the bottom tray 8 may be configured to securely fit over all of an individual's lower teeth, or over just a portion of an individual's lower teeth.

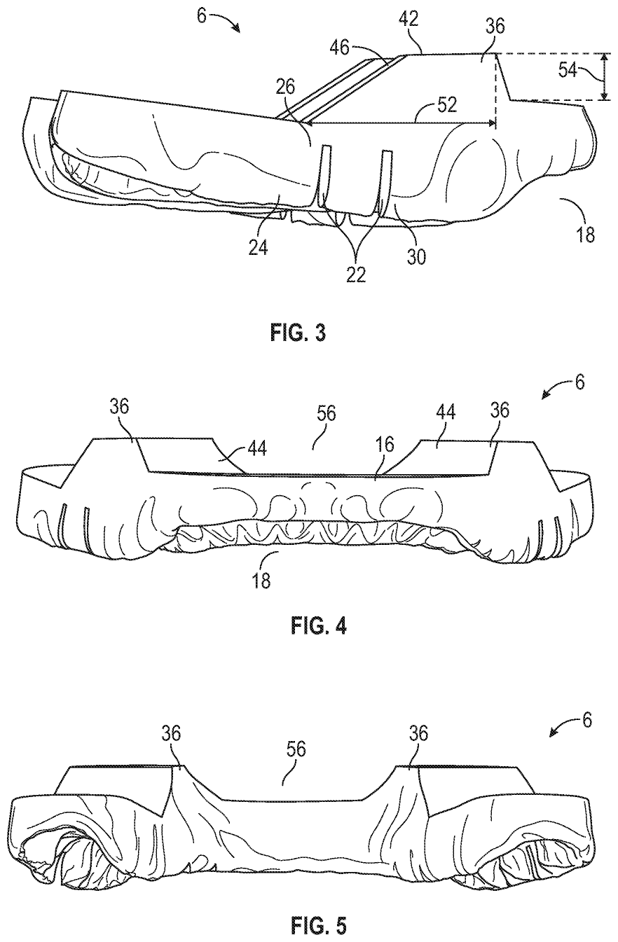

Still referring to FIG. 2, the lower portion 6 may include a lower shell 20. The lower shell 20 may cover the bottom tray 8 and/or offer support to the bottom tray 8. Inasmuch as the bottom tray 8 is within the lower shell 20, the lower shell may be considered as receiving the user's lower teeth. In various implementations, the lower shell 20 may correspond to the structure of an individual's mouth. In such implementations, the shape, size, position, and the thickness of the shell may be individualized to ensure a comfortable and precise fit of the lower portion 6 within a user's mouth. In various implementations, the shell 20 may include a recess in a front sidewall of the lower shell forming a frontal space 18 through which an individual's lower teeth might be exposed when the lower portion 6 is coupled over an individual's lower teeth. The frontal space 18 can also be clearly seen in FIG. 3, which illustrates a side view of the lower portion 6 of a sleep aid device, and FIG. 4, which illustrates a front view of the lower portion 6 of a sleep aid device.

Referring to FIGS. 2-3, in various implementations the shell 20 may include one or more slots 22 extending from a lower portion 24 of the shell 20 towards an upper portion 26 of the shell. The slot may extend through the surface matching facial structure. In various implementations, the lower portion 6 (or any other lower portion disclosed herein) may include two slots on a first side 28 of the lower portion 6 and two slots on a second opposing side 30 of the lower portion 6. In other implementations, the lower portion 6 may include more or less than four slots and they may be located in varying locations on the lower portion, including in the lingual matching surface 12. In various implementations, the slots may be used to hold an elastic 32 which may be used to hold the lower portion 6 and the upper portion 4 together. As disclosed later herein, the elastic may also be held in place by one or more slots in the upper portion 4.

Referring to FIG. 2, the lower portion 6 may include a lower bar 34. The bar 34 may include material located over the lower shell 20 and may help provide the desired occlusal clearance for the sleep aid device 2. In various implementations, the lower bar 34 of the lower portion 6 may be thicker to provide for more material on top of the lower shell 20, and in turn, more occlusal clearance. In other implementations the bar 34 may be thinner to provide for less occlusal clearance. In various implementations the lower bar 34 may be coupled over the lower shell 20. Alternatively, in other implementations the lower bar 34 may be included in the lower shell 20 and may form the upper portion of the lower shell 20.

In various implementations, the lower portion 6 may include one or more lower projections 36. In implementations with only a single projection, the projection may extend around the front of the sleep aid device. In other implementations, and as illustrated by FIG. 2, the lower portion 6 may include a first lower projection 38 and a second lower projection 40. In particular implementations, the one or more projections may be part of the lower bar 34 or may be separate from and coupled to the bar. As illustrated by FIG. 2, the lower projections 36 are formed with the lower bar 34 as a single uniform piece. In other implementations, the lower projections 36 may be either fixedly coupled to the lower bar 34 or may be removably coupled to the lower bar. The lower projections 36 may be of any shape and size. As illustrated by FIG. 3, the lower projections 36 may each have a length 52 and a height 54. In various implementations, the height 54 of the lower projections 36 may be greater than the height of the upper projections 90. As disclosed herein, the first lower projection 38 and the second lower projection 40 are configured to either shift forward a user's mandible, provide and/or increase occlusal clearance for a user, or both shift the mandible forward while also increasing the space between the user's upper and lower jaw. Accordingly, in implementations such as the one illustrated by FIG. 3, the length 52 of the lower portions 36 may determine the amount the user's jaw is shifted forward while the height 54 of the lower portions 36 may determine the amount of occlusal clearance. In various implementations, and as illustrated by FIG. 2, the lower projections 36 may have a flat top surface 42. The front surface 44 and the rear surface 46 of each of the lower projections 36 may be sloped. In other implementations, the front surface 44 and/or the rear surface 46 may be substantially perpendicular to the top surface 42 of the lower projections 36. In particular implementations, though not illustrated, the front surface 44 and/or rear surface 46 of the lower projections 36 may include an inverted slope. The sides 48 of the lower projections 36 may or may not be coextensive with the outer surface of the facial matching surface 14 of the lower shell 20 and the lingual matching surface 12 of the lower shell. At least a portion of the one or more projections 36 may be configured to correspond and intersect with a portion of one or more projections on an upper portion 4 of a sleep aid device 2. As illustrated by FIG. 3, the rear surfaces 46 of the lower projections 36 and the top surfaces 42 of the lower projections are illustrated as straight and smooth surfaces. In other implementations, either one of these surfaces, or both of these surfaces, may include notches, ridges, cavities, bumps, patterns, or other protrusions configured to correspond with surfaces of the upper portion in order to better secure the lower portion to the upper portion when used by a user. Similarly, the lower projections 36 have particular slopes, as illustrated by FIG. 3. These slopes of the rear, top, front, and sidewalls may all be varied, along with corresponding slopes of the upper portion, in order to move a user's jaw the predetermined amount and to the predetermined location. In various implementations, an entirety of the top surfaces 42 of the lower projections 36 may be configured to contact the upper bar 88 of the upper portion 4.

In various implementations, the lower portion 6 may also include an intersecting area 50 on a top surface of the lower portion 6 which is configured to intersect and contact the upper portion 4. The intersecting area may be flush with a projection of the upper portion, as described later herein. In various implementations, the upper surface of the lower bar 34, the top 42 of the lower projections 36, the rear surfaces 46 of the lower projections, and any combination thereof, may be an intersecting area inasmuch as any of these, or all of these, areas may be configured to intersect and contact a portion of the upper device 4.

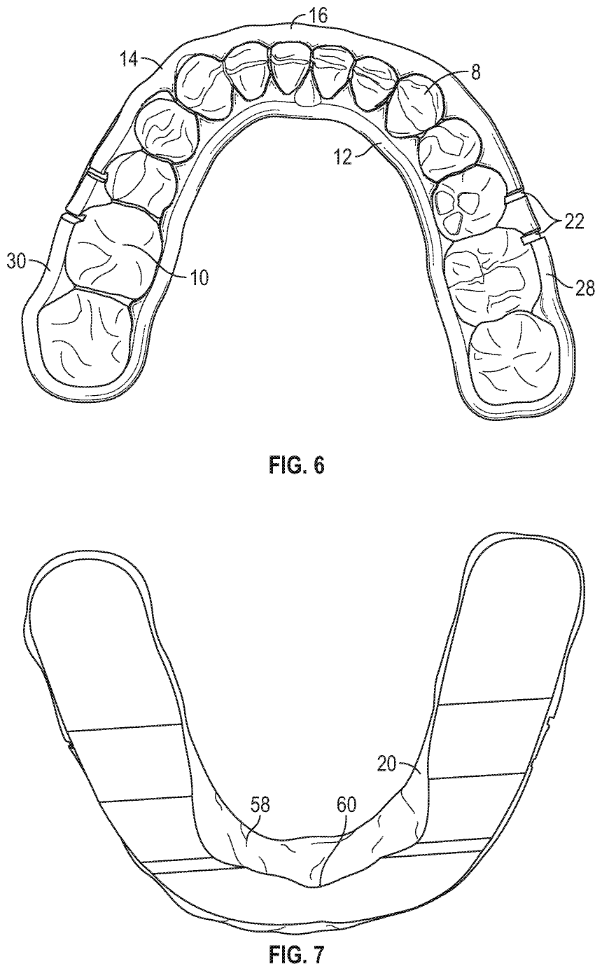

Referring to FIG. 4, a front view of a lower portion of a sleep aid device is illustrated, and referring to FIG. 5, a rear view of a lower portion of a sleep aid device is illustrated. As illustrated by FIGS. 4-5, the front 16 of the lower portion 6 may include a recess 56 formed between the lower projections 36. In various implementations, the front surfaces 44 of the lower projections 36 may form the sidewalls of the recess 56. In such implementations, the recess 56 may facilitate breathing of a user inasmuch as it provides a passageway for air. Further, in various implementations, the recess 56 may also provide space for the tongue of a user when the sleep aid device 2 is in use.

Referring to FIG. 7, a top view of a lower portion of a sleep aid device is illustrated. As illustrated by FIG. 7, the outer lingual surface 58 of the lower shell 20 may include a divot 60 near the front of the lower portion 6. In such implementations, the divot may provide additional room for a user's tongue when the sleep aid device 2 is used by a user.

As the one or more projections 36 of the lower portion 6 intersect with the one or more projections of the upper portion 4, the lower jaw, or mandible, may be repositioned. In various implementations, the lower jaw may be repositioned in a forward or horizontal manner and/or in a vertical manner. By altering the size and/or shape of the projections, the amount of repositioning done by the sleep aid device may be altered or customized.

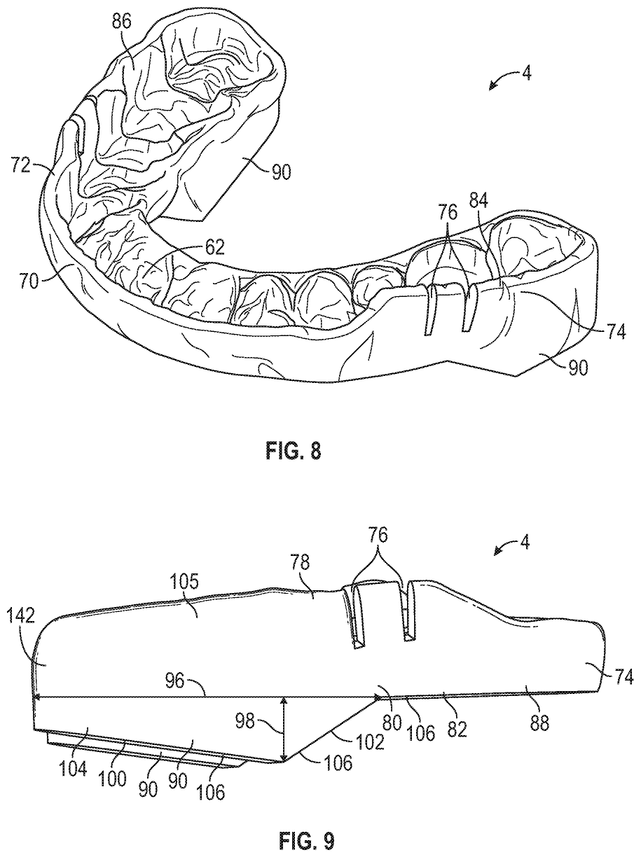

Referring to FIG. 8, a top perspective view of an upper portion of a sleep aid device is illustrated. In various implementations the upper portion 4 may be a single unitary piece without any kind of seam. In other implementations, the upper portion 4 may be made from various portions which are combined together or separately formed and combined together. In various implementations, the upper portion may be made from any material previously disclosed herein. In various implementations, the upper portion 4 may be a single material, while in other implementations differing materials may be included in the upper portion 4 either in various sections or portions of the device or through layering of different materials.

Referring to FIG. 12, a top view of the upper portion is illustrated. In various implementations, the upper portion 4 may include an upper tray 62. The upper tray may be a portion of the upper portion 4 molded to fit over an individual's upper teeth. In various implementations, the upper tray may be made to specifically match a single user's upper teeth. In various implementations, the upper tray 62 may include, by non-limiting example, a crown matching surface 64 configured to match the crowns of an individual's lower teeth, a lingual matching surface 66 configured to match the lingual structure of an individual's lower teeth, a facial matching surface 68 configured to match the facial structure of an individual's lower teeth, and any combination thereof. In various implementations, the upper tray 4 may match the crowns of an individual's upper teeth, the lingual surfaces of an individual's upper teeth, the facial surfaces of an individual's upper teeth, and any combination thereof. As illustrated by FIG. 8, the front 70 of the upper portion 4 may include a recess is a front sidewall of the upper shell 74 forming a frontal space 72 where the upper tray 62 does not include a corresponding facial matching surface 68 covering the entirety of the front teeth. This frontal space 72 can also be seen in FIG. 10, which is a front view of the upper portion of the sleep aid device, as well as in FIG. 11, which is an illustration of a rear view of the lower portion of the sleep aid device. The upper tray may be configured to securely fit over all of an individual's upper teeth, or over just a portion of an individual's upper teeth.

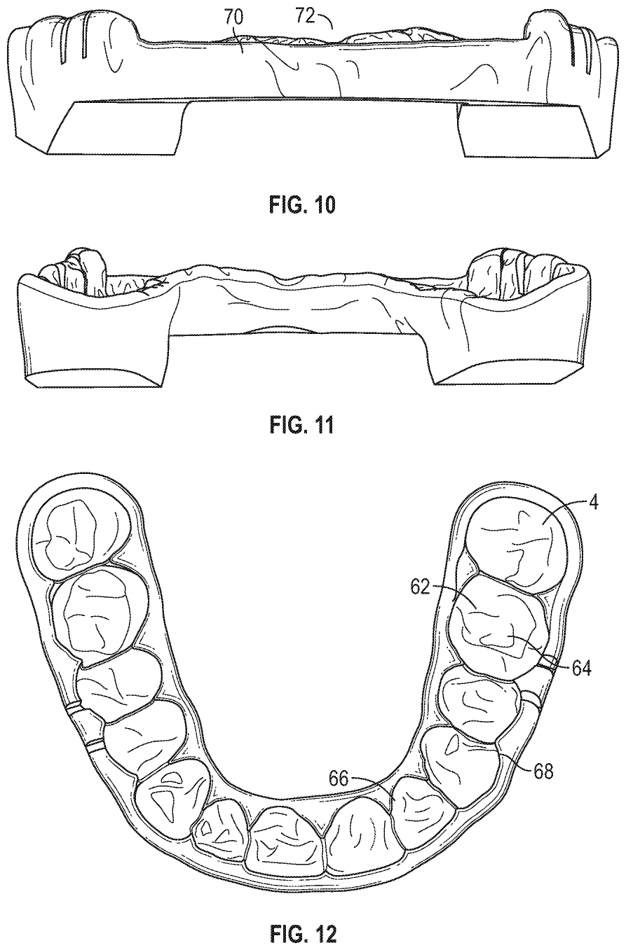

Referring back to FIG. 8, the upper portion 4 may include an upper shell 74. The upper shell 74 may cover the upper tray 62 and/or offer support to the upper tray 62. Accordingly, the upper shell 74 may be considered as receiving the user's upper teeth. In various implementations, the upper shell 74 may correspond to the structure of an individual's mouth. In such implementations, the shape, size, position, and the thickness of the upper portion 4 may be individualized to ensure a comfortable and precise fit of the upper portion within a user's mouth. In various implementations, the upper shell 74 may include a frontal space 72 through which an individual's upper teeth might be exposed when the upper portion 4 is over an individual's upper teeth. In various implementations, the upper shell 74 may include one or more slots 76 extending from an upper portion 78 of the shell towards the lower portion 80 of the shell, or towards the bar 82 of the upper portion 4. This is clearly illustrated by FIG. 9, which illustrates a side view of the upper portion of the sleep aid device. The slots 76 may extend through the facial matching surface 68. In various implementations, the upper portion 4 may include two slots on a first side 84 of the upper portion and two slots on a second opposing side 86 of the upper portion. In other implementations, the upper portion may include more or less than four slots and they may be located in varying locations on the upper portion, including in the lingual matching surface 66. In various implementations, the slots may be used to secure an elastic 32 which may be used to hold the upper portion and the lower portion together, as illustrated by FIG. 1. As disclosed previously herein, the elastic may also be held in place by one or more slots in the lower portion. In other implementations, coupled mechanisms aside from the elastics may be used. Such mechanisms may include, by non-limiting example, magnets, a pressure fit, or any other coupling mechanism between the lower portion 6 and the upper portion 4.

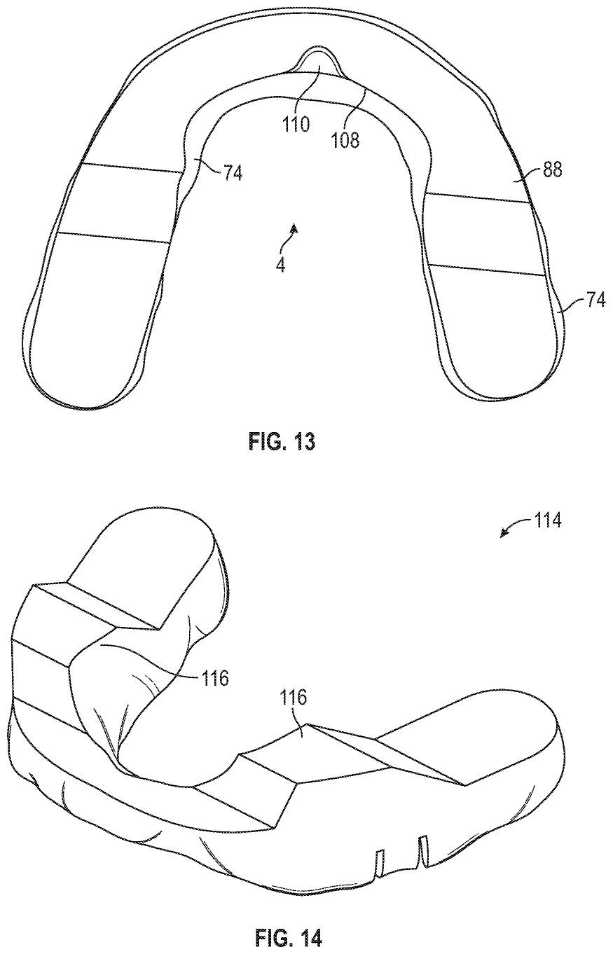

Referring to FIG. 13, a bottom view of an upper portion of a sleep aid device is illustrated, and referring to FIG. 9, a side view of an upper portion of a sleep aid device is illustrated. In various implementations, and as illustrated by FIGS. 9 and 13, the upper portion 4 may include an upper bar 88 coupled below the upper shell 74. The upper bar 88 may be material located below the shell 74 and may provide, together with the bar and/or projections of the lower and upper portions, the desired occlusal clearance for the sleep aid device. In various implementations, the upper bar 88 of the upper portion 4 may be thicker to provide for more occlusal clearance, while in other implementations the upper bar may be thinner to provide for less occlusal clearance.

Referring to FIG. 8 and FIG. 9, the upper portion 4 may include one or more upper projections 90. As illustrated by FIG. 8, in particular implementations the upper portion 4 includes a first upper projection 92 and a second upper projection 94 extending towards the lower bar of the lower portion 6. The first upper projection and the second upper projection extend from a rear portion 142 of the upper portion 4. In particular implementations, the one or more projections 90 may be part of the upper bar 88 or may be separate from and coupled to the bar. As illustrated by FIGS. 8 and 9, the upper projections 90 are formed with the upper bar 88 as a single uniform piece. In other implementations, the upper projections 90 may be either fixedly coupled to the upper bar 88 or may be removably coupled to the upper bar. The upper projections 90 may be of any shape and size. As illustrated by FIG. 9, the upper projections 90 may each have a length 96 and a height 98. As disclosed herein, the first upper projection 92 and the second upper projection 94 are configured to either shift forward a user's mandible, provide and/or increase occlusal clearance for a user, or both shift the mandible forward while also opening the user's mouth. Accordingly, in implementations such as the one illustrated by FIG. 9, the length 96 of the upper portions 90 may determine the amount the user's jaw is shifted forward while the height 98 of the upper portions 90 may determine the amount of occlusal clearance. In various implementations, and as illustrated by FIG. 9, the upper projections 90 may have a planar bottom surface 100. The bottom surface may be flat or sloped. In various implementations, an entirety of the planar bottom surface may be configured to contact the upper surface, or the lower bar of the lower portion 6. The front surface 102 of each of the lower projections 36 may be sloped. In other implementations, the front surface 102 may be substantially perpendicular to the bottom surface 100 of the upper projections 90. In particular implementations, though not illustrated, the front surface 102 of the upper projections 90 may include an inverted slope. The sides 104 of the upper projections 90 may or may not be coextensive with the outer surface 105 of the facial matching surface 68 of the upper shell 74 and the outer surface of the lingual matching surface 66 of the upper shell. At least a portion of the one or more projections 90 may be configured to correspond and intersect with a portion of one or more projections on the lower portion 6 of a sleep aid device 2. As illustrated by FIG. 9, the front surfaces 102 of the upper projections 90 and the bottom surfaces 100 of the upper projections are illustrated as straight and smooth surfaces. In other implementations, either one of these surfaces, or both of these surfaces, may include notches, ridges, cavities, bumps, patterns, or other protrusions configured to correspond with surfaces of the lower portion in order to better secure the lower portion to the upper portion when the sleep aid device is used by a user. Similarly, the upper projections 90 have particular slopes, as illustrated by FIG. 9. These slopes of the bottom, front, and sidewalls may all be varied, along with corresponding slopes of the lower portion, in order to move a user's jaw the predetermined amount and to the predetermined location.

In various implementations, the upper portion 4 may also include an intersecting area 106 on a bottom surface of the upper portion 4 which is configured to intersect and contact the lower portion 6. In various implementations, the intersecting area 106 of the upper portion and the intersecting area 50 of the lower portion 6 may be flush with one another. In various implementations, the portion of the bottom surface, adjacent the upper projections, of the upper bar 88, the bottom surface 100 of the upper projections 90, the front surfaces 102 of the upper projections, and any combination thereof, may be an intersecting area inasmuch as any of these, or all of these, areas may be configured to intersect and contact a portion of the lower device 6.

Referring to FIG. 13, a bottom view of an upper portion of a sleep aid device is illustrated. As illustrated by FIG. 13, the outer lingual surface 108 of the lower shell 74 may include a divot 110 near the front of the upper portion 13.

Referring back to FIG. 1, the rear surfaces 46 of the lower projections 36 are configured to contact the front surfaces 102 of the upper projections 90. As the one or more projections 90 of the upper portion 4 intersect with the one or more projections 36 of the lower portion 6, the lower jaw, or mandible, may be repositioned. In various implementations, the lower jaw may be repositioned in a forward or horizontal manner and/or in a vertical manner. By altering the size and/or shape of the upper or lower projections, the amount of repositioning done by the sleep aid device may be altered or customized in either a horizontal or vertical matter. Particular implementations of the sleep aid device 2 may only shift the mandible forward. Other particular implementations of the sleep aid device 2 may shift the mandible downward, or increase the occlusal clearance of the user. Still other particular implementations may simultaneously shift the user's mandible forward while also increasing the vertical distance between the user's top and bottom teeth. Similarly, by altering the thicknesses of the bars the lower and upper portions the occlusal clearance may be customized for the user. Further, as illustrated by FIG. 1, the lower portion 6 and the upper portion 4 may be configured to form an opening 112 at the front of the device 2 when the lower portion 6 is coupled to the upper portion 4.

In various implementations, by adjusting a user's mandible using the sleep aid device 2 illustrated by FIGS. 1-13, the device may be able to prevent a user's soft tissue, including the user's tongue, from blocking the user's airway. Accordingly, the sleep aid device of FIGS. 1-13 may prevent OSA.

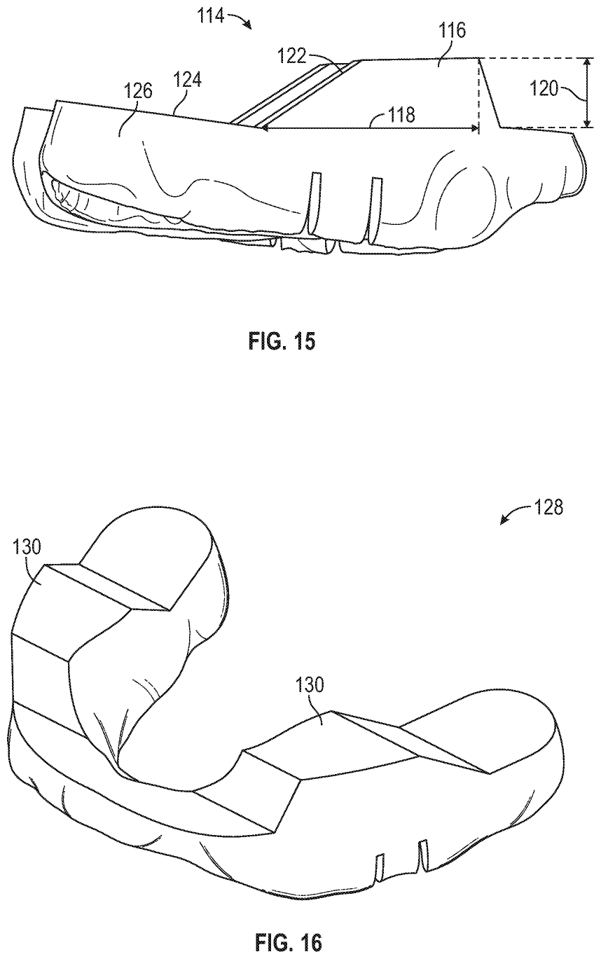

Referring to FIG. 14, a top perspective view of a second implementation of a lower portion of a sleep aid device is illustrated, and referring to FIG. 15, a side view of a second implementation of a lower portion of a sleep aid device is illustrated. FIGS. 14-15 are very similar to FIGS. 2 and 3, with the difference being that FIGS. 14-15 illustrate lower portions 114 having lower projections 116 having larger projections. In various implementations, a length 118 of the lower projections 116 and/or a height 120 of the lower projections may be greater than a corresponding length and height of the lower projections of FIGS. 2 and 3. Accordingly, in implementations having a greater length, the increased length of the projections 116 may result in an increased forward movement of the mandible when the lower portion 114 is coupled to the upper portion 4 as compared to when the lower portion 6 is coupled to the upper portion 4. Similarly, in implementations having a greater height, the increased height of the projections 116 may result in increased occlusal clearance when the lower portion 114 is coupled to the upper portion 4 as compared to when the lower portion 6 is coupled to the upper portion 4. In various implementations, the slope of the rear surface 122 of the lower projections 116 may be the same as or different from the slope of the rear surface 46 of the lower projections 36. In other implementations, the lower portion 114 may result in increased occlusal clearance as a result of a thicker bar 124 coupled over the lower shell 126. By comparing FIG. 3 to FIG. 15, the thicker bar, the increased height 120 of the projection 116, and the increased length 118 of the projection is visible and all will clearly lead to increased occlusal space and increased forward positioning of the lower jaw.

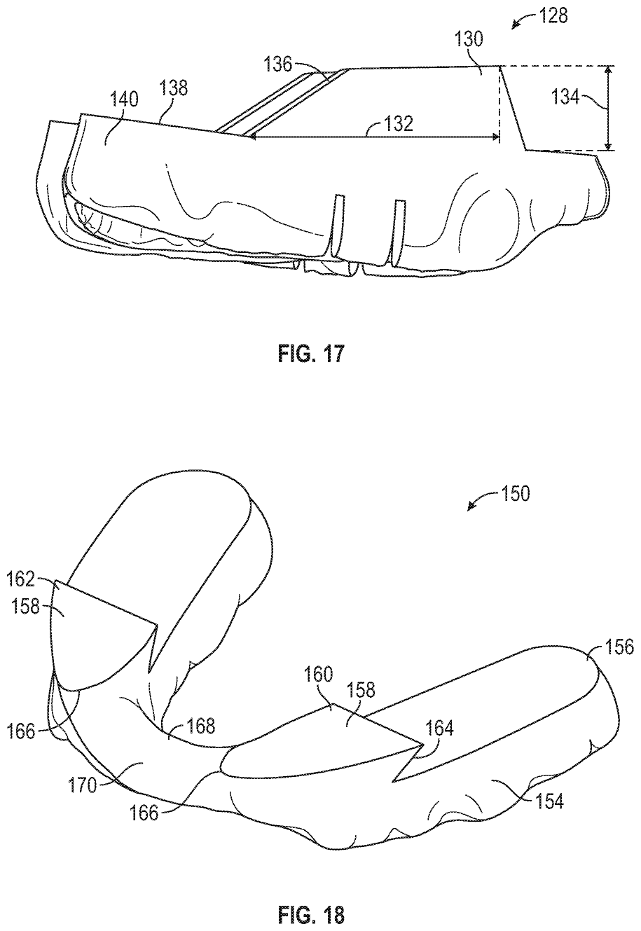

Referring to FIG. 16, a top perspective view of a third implementation of a lower portion of a sleep aid device is illustrated, and referring to FIG. 17, a side view of a second implementation of a lower portion of a sleep aid device is illustrated. FIGS. 16-17 are very similar to FIGS. 14-15, with the difference being that FIGS. 16-17 illustrate lower portions 128 having lower projections 130 having larger projections. In various implementations, a length 132 of the lower projections 130 and/or a height 134 of the lower projections may be greater than a corresponding length and height of the lower projections of FIGS. 14-15. Accordingly, in implementations having a greater length, the increased length 132 of the projections 130 may result in an increased forward movement of the mandible when the lower portion 128 is coupled to the upper portion 4 as compared to when the lower portion 114 is coupled to the upper portion 4. Similarly, in implementations having a greater height, the increased height 134 of the projections 130 may result in increased occlusal clearance when the lower portion 128 is coupled to the upper portion 4 as compared to when the lower portion 114 is coupled to the upper portion 4. In various implementations, the slope of the rear surface 136 of the lower projections 128 may be the same as or different from the slope of the rear surface 122 of the lower projections 116. In other implementations, the lower portion 128 may result in increased occlusal clearance as a result of a thicker bar 138 coupled over the lower shell 140. By comparing FIG. 17 to FIG. 15, the thicker bar, the increased height 134 of the projection 130, and the increased length 132 of the projection is visible and all will clearly lead to increased occlusal space and increased forward positioning of the lower jaw.

Referring to FIGS. 18-29, another implementation of a sleep aid device similar to the sleep aid device of FIG. 1 is illustrated. Referring specifically to FIG. 18, a top perspective view of a fourth implementation of a lower portion of a sleep aid device is illustrated. In various implementations, the lower portion 150 may include a lower tray 152 (as illustrated by FIG. 20), a lower shell 154, and a lower bar 156. The lower tray 152, the lower shell 154, and the lower bar 156 may be the same as or similar to any lower tray, lower shell, and lower bar disclosed herein. As illustrated, the lower portion 150 also includes lower projections 158. The lower projections 158 protrude upward from the lower bar 156 (in implementations where the projections are formed over the lower bar). In particular implementations, the lower portion 150 includes a first lower projection 160 and a second lower projection 162. Contrary to the lower portions of FIGS. 1-7 and 14-17, the projections 158 include rear surfaces 164 having an inverted slope, or a slope extending to the front of the device from the top of the projections 158. In such an implementation, the lower projections 158 may interlock with the upper projections of the upper portion. The lower projections 158 may include varying lengths and heights, similar to the other implementations of lower projections disclosed herein. Accordingly, the lower projections 158 may interact with upper projections of an upper portion in order to shift a user's lower jaw or mandible forward. The lower projections 158 may also interact with the upper portion to increase the vertical distance, or the occlusal clearance, between a user's upper and lower jaw. In particular implementations, the lower portion 150 may only shift the user's mandible forward, may only increase the occlusal clearance, or may both shift the user's mandible forward and increase the occlusal clearance of the user.

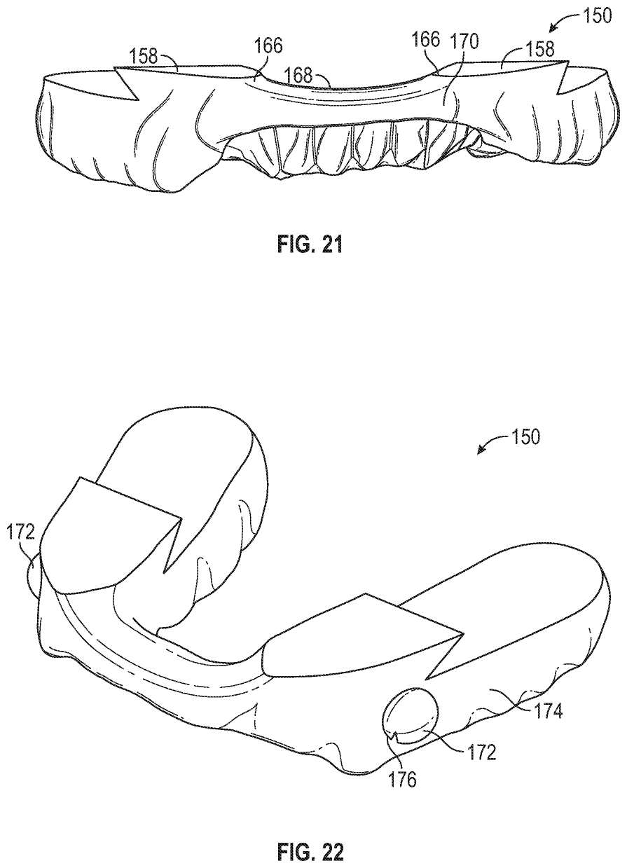

Still referring to FIG. 18, the lower projections may include front surfaces 166. Referring to FIG. 21, a front view of a fourth implementation of a lower portion is illustrated. As illustrated, in various implementations the front surface may be have a minimal height and a gradual slope, leading to a shallow recess 168 in the front 170 of the lower portion 150. In other implementations, the lower projections may not include a recess between the lower projections. Accordingly, in such implementations the lower portion could be considered to have a single projection which extends around the front of the device. In such implementations, the single projection may have holes therethrough to allow air to pass through when a user is using the sleep aid device.

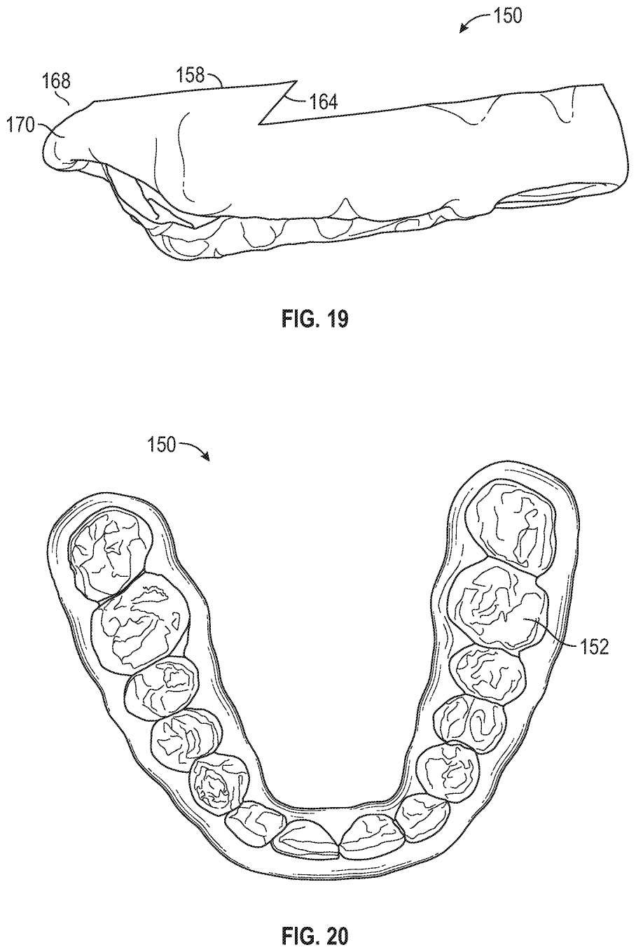

Referring to FIG. 19, a side view of the fourth implementation of the lower portion of the sleep aid device is illustrated. FIG. 19 clearly illustrates the angled rear surface 164 of the lower projection 158. In various implementations, the angle may be more or less steep than what is illustrated by FIG. 19 in order to provide a more or less secure interlock with the upper portion.

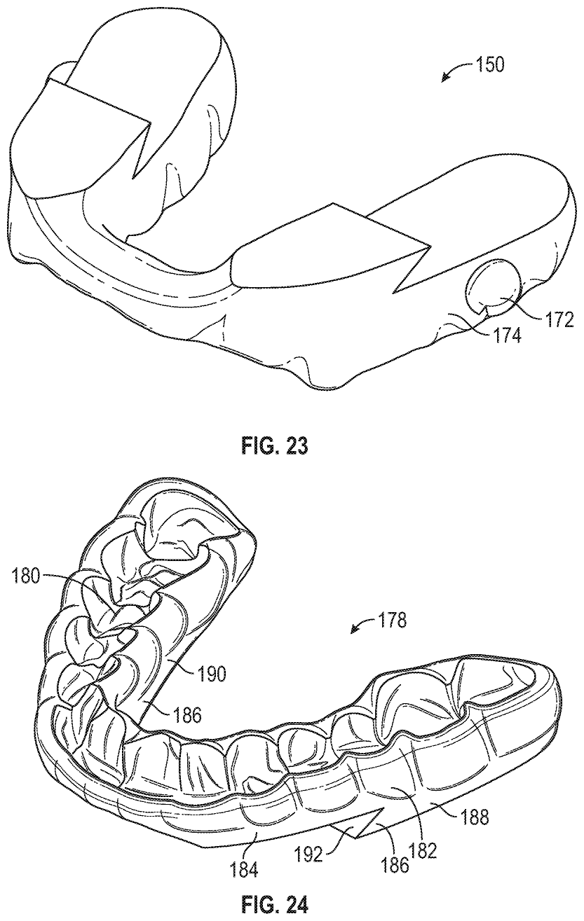

Referring to FIG. 22, a top perspective view of a fourth implementation of a lower portion of a sleep aid device with a first implementation of an attachment is illustrated. In various implementations one or more attachment portions 172 may be coupled to one or more sidewalls 174 of the lower portion 150. In various implementations, the attachment portions 174 may be rounded or smooth in order to reduce irritation of a user using the sleep aid device. In various implementations, the attachment portion may include a notch 176 in the lower surface of the attachment portion 172. The notch 176 may allow for an elastic to couple to the attachment portion 172 and couple to a corresponding attachment mechanism of the upper portion in order to secure the lower and upper portion together. Referring to FIG. 23, a top perspective view of a fourth implementation of a lower portion of a sleep aid device with a second implementation of an attachment is illustrated. As illustrated by FIG. 23, the lower portion 150 may include the attachment portion 172 in various positions along the sidewall 174. The attachment portion 172 may be fixed to the lower portion 150 in a position which provides the most comfort to a user when using the sleep aid device.

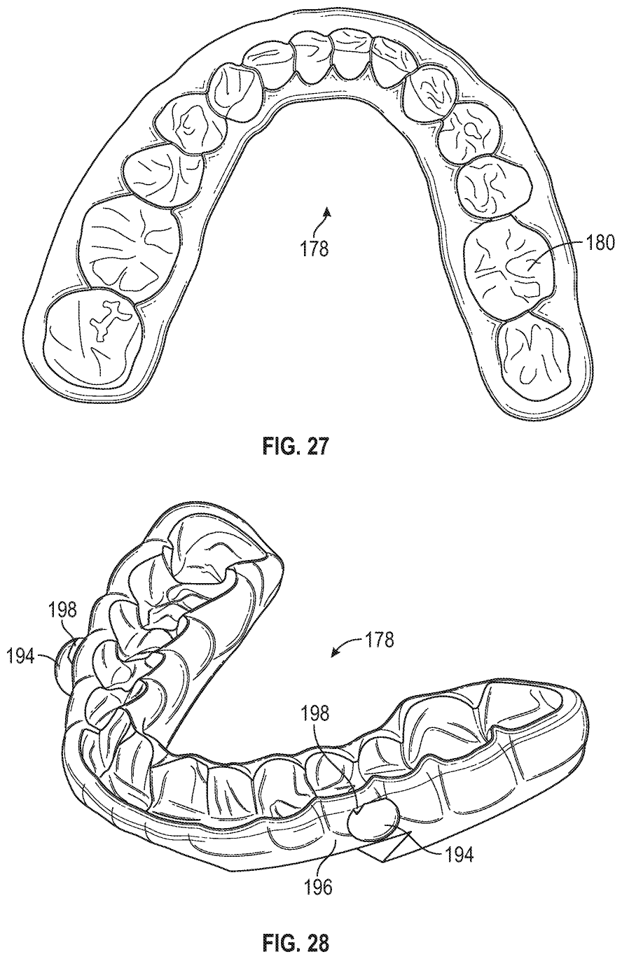

Referring to FIG. 24, a top perspective view of a second implementation of an upper portion of a sleep aid device is illustrated. In various implementations, the upper portion 178 may include an upper tray 180 (as further illustrated by FIG. 27), an upper shell 182, and an upper bar 184. The upper tray 180, the upper shell 182, and the upper bar 184 may be the same as or similar to any upper tray, upper shell, and upper bar disclosed herein. As illustrated, the upper portion 178 also includes upper projections 186. The upper projections 186 protrude downward from the upper bar 184 (in implementations where the projections are formed under the upper bar). In particular implementations, the upper portion 178 includes a first upper projection 188 and a second upper projection 190. Contrary to the upper portions of FIGS. 1 and 8-13, the projections 186 include front surfaces 192 having an inverted slope, or a slope extending to the rear of the device from the bottom of the projections 186. In such an implementation, the upper projections 186 may interlock with the lower projections 158 of the lower portion 150. The upper projections 186 may include varying lengths and heights, similar to the other implementations of upper projections disclosed herein. Accordingly, the upper projections 186 may interact with lower projections 158 of a lower portion 150 in order to shift a user's lower jaw or mandible forward. In various implementations, the upper projections 186 may also interact with the lower portion 150 to increase the vertical distance, or the occlusal clearance, between a user's upper and lower jaw.

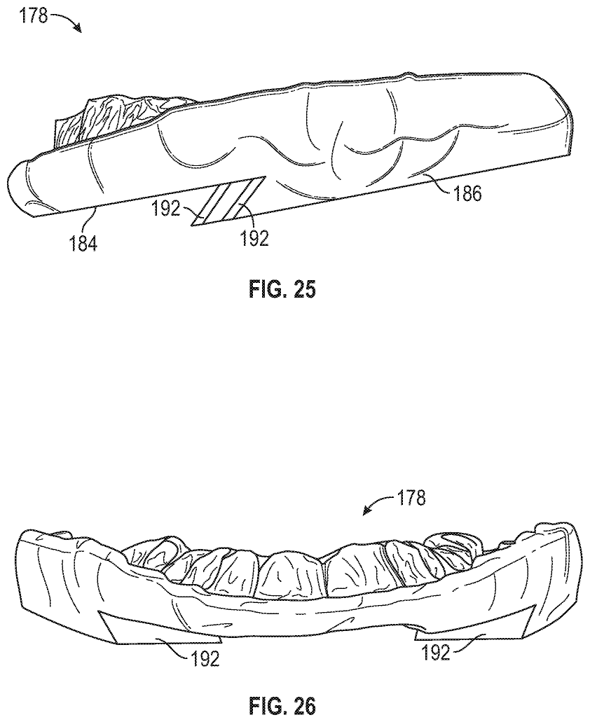

Referring to FIG. 25, a side view of a second implementation of an upper portion of a sleep aid device is illustrated, and referring to FIG. 26, a front view of a second implementation of an upper portion of a sleep aid device is illustrated. FIG. 25 clearly illustrates the angled front surface 192 of the upper projection 186. In various implementations, the angle may be more or less steep than what is illustrated by FIG. 25 in order to provide a more or less secure interlock with the upper portion.

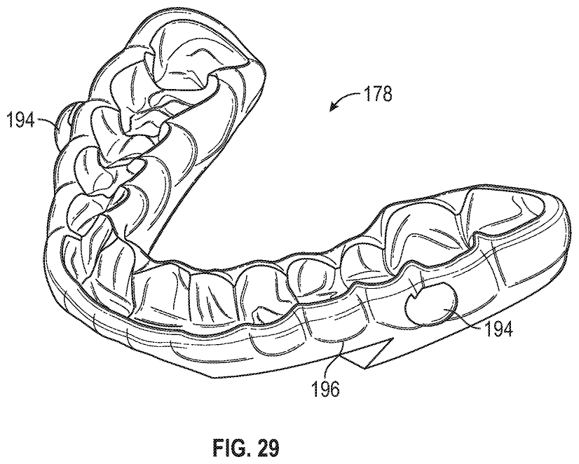

Referring to FIG. 28, a top perspective view of a second implementation of an upper portion of a sleep aid device with a first implementation of an attachment is illustrated. In various implementations one or more attachment portions 194 may be coupled to one or more sidewalls 196 of the upper portion 178. In various implementations, the attachment portions 194 may be rounded or smooth in order to reduce irritation of a user using the sleep aid device. In various implementations, the attachment portion 194 may include a notch 198 in the upper surface of the attachment mechanism 194. The notch 198 may allow for an elastic to couple to the attachment mechanism 194 and couple to a corresponding attachment mechanism of the lower portion 150 in order to secure the lower and upper portion together. Referring to FIG. 29, a top perspective view of a second implementation of an upper portion of a sleep aid device with a second implementation of an attachment is illustrated. As illustrated by FIG. 29, the upper portion 178 may include the attachment portion 194 in various positions along the sidewall 196. The attachment portion 194 may be fixed to the upper portion 178 in a position which provides the most comfort to a user when using the sleep aid device.

While the implementations illustrated herein depict sleep aid devices where the rear surfaces of the lower projections intersect with the front surfaces of the upper projections, it is understood that the projections of the upper and lower portions may intersect in a variety of ways. In various implementations, the projection of the lower portion may include a depression therein configured to receive a projection of the upper portion. Alternatively, the projection of the upper portion may include a depression therein configured to receive a projection of the lower portion. In other implementation, only either the upper or the lower portion may include a projection configured to fit within a recess of either the upper or lower portion. Any other configuration of projections of the upper and lower portions of a sleep aid device may be implemented in the implementations disclosed herein.

In various implementations a sleep aid device kit may include a single upper portion and a single lower portion. The sleep aid device of the sleep aid device kit may be customized to fit within an individual's mouth and reduce the apnea-hypopnea index (AHI) (or the number of apnea or hypopnea events during sleep due to an obstruction of the airway) of the individual by repositioning the jaw.

In other implementations, the sleep aid device kit may include an upper portion and multiple lower portions, each lower portion including a different shaped and/or sized bar and/or lower projections. In such implementations, the upper portion may be any type of upper portion disclosed herein. The kit may also include a minimum lower portion. The minimum lower portion may include lower projections having the shortest length and/or height. The lower projections may also include the thinnest bar. The minimum lower portion may be similar to or the same as the lower portion of FIGS. 2-7. In various implementations, the method of repositioning a user's jaw may include first using the sleep aid device with the minimal lower portion. Over time, the user may exchange the lower portion for other lower portions with greater occlusal clearances, projections, and/or other physical parameters to continue to adjust the position of the lower jaw until the position of the lower jaw has been corrected and/or the user's AHI has been reduced.

In various implementations, the kit may include an intermediate lower portion. The intermediate lower portion may include lower projections having a length and/or height greater than the length and/or height of the minimum lower portion. In various implementations, the height of the lower projections of the intermediate lower portion may be 2 mm greater than the height of the lower projections of the minimum lower portion. In other implementations, the height of the lower projections of the intermediate lower portion may be more than or less than 2 mm greater than the height of the lower projections of the minimum lower portion. The lower portions may also include a lower bar having a thickness greater than the lower bar of the minimum lower portion. The intermediate lower portion may be similar to or the same as the lower portion of FIGS. 14-15. In various implementations, the method of repositioning a user's jaw may include using the sleep aid device after the sleep aid device has been used by a user with the minimum lower portion.

The kit may also include a maximum lower portion. The maximum lower portion may include lower projections having the greatest length and/or height. In various implementations, the height of the lower projections of the maximum lower portion may be 2 mm greater than the height of the lower projections of the intermediate lower portion. In other implementations, the height of the lower projections of the maximum lower portion may be more than or less than 2 mm greater than the height of the lower projections of the intermediate lower portion. The lower projections may also include a thicker bar. The maximum lower portion may be similar to or the same as the lower portion of FIGS. 16-17. In various implementations, the method of repositioning a user's jaw may include using the sleep aid device with the maximum lower portion after the sleep aid device was used with the intermediate lower portion.

In various implementations, the sleep aid device kit may include a second intermediate lower portion having lower projections with a length and/or height greater than the length and/or height of the intermediate lower portions but less than the length and/or height of the maximum lower portions.

In other implementations, the sleep aid device may similarly include multiple upper portions and a single lower portion, or multiple upper portions and multiple lower portions to adjust and customize the correction of the position of the jaw.

By including multiple lower portions and/or multiple upper portions, a user may gradually reposition their jaw until the optimal position and results are achieved. In particular implementations, the sleep aid device kit may include one upper portion and multiple lower portions, such as two, three, four, five, or more lower portions, including any type of upper and lower portion disclosed herein having the same or varying sized upper or lower projections. By including multiple lower portions, a user may gradually reposition their lower jaw, such as by advancing their lower jaw forward, increasing their occlusal clearance, or both advancing the jaw and increasing occlusal clearance, until the optimal position and results are achieved. This is done by having the user first use the lower portion that most minimally adjusts the user's jaw. Over time, the user gradually replaces the lower portion with other lower portions that increasingly adjust the users jaw. In various implementations, each lower portion may increase the occlusal clearance by an additional 2 mm from the previous lower portion used, resulting in a gradual change of the positioning of the users jaw over time done in 2 mm increments. Other implementations may include incremental changes greater than or less than 2 mm. The user uses the lower portion that most significantly changes the lower jaw, and in turn adjusts the user's jaw to the desired position, after all other lower portions have been used. This process may also permit the user to be more comfortable when using the device, as the jaw's position is gradually moved to the optimal position rather than immediately moved. In implementations having multiple lower portions, the upper projections may contact the lower bar of the all of the lower portions of the kit. In such implementations, each bar of each lower portion may correspond to the height of the lower projections enabling the a precise and complete fit between the upper portion and the lower portion, including a precise and complete fit between the upper projections and the lower bar.

In other implementations, a sleep aid device kit may include a single upper portion and a single lower portion with adjustable projections extending from the upper and/or lower portion. By being able to adjust either the size of the projections, or the position of the projections, the lower jaw may be adjusted similar to the methods described above.

In various implementations, a method for making the sleep aid device described in FIGS. 1-17 may include taking a digital impression or a scan of a user's lower teeth and upper teeth. The method also includes determining using the impression or scan how the jaw should be repositioned or corrected.

In various implementations, various software programs may be used to produce the sleep aid device. In a particular implementation, software marketed under the tradename 3SHAPE® by 3Shape A/S of Copenhagen, Denmark, may be used. In various implementations, the method may include using the software and preparing a digital 3D model-set (or digital lower tray and upper tray) by putting bases on the digital scan impressions of the lower teeth and the top teeth. The software may also be used to clean/correct any defects from the scans. In various implementations, the software may include a virtual articulator. In such implementations, the method may include using the virtual articulator to put the model sets in a desired position based upon measurements taken by a practitioner to create a device that can correspondingly adjust the position of the jaw.

In various implementations, the method includes using the software to create a digital model of a lower shell over the model set of the lower portion and a digital model of an upper shell over the model set of the upper portion. The digital model of the shells may be customized to include certain offsets, retentions, sizes, shapes, positions, and thicknesses to provide a comfortable and precise fit for a specific user. In various implementations, the method may also include designing a digital model of a lower bar to go over the lower shell of the lower portion and a digital model of an upper bar to go over (or below, depending on the position of the 3D model) the shell of the upper portion. The digital models of the bars may then be adjusted to obtain the desired occlusal clearance. The bars may or may not also include the projections. In implementations where the bar is separate from the bar, the method may include forming one or more digital models of projections over the bar and/or shell. An implementation of the method may include combining the digital model of the lower shell and the digital model of the lower bar (and a digital model of the one or more lower projections, if not included with the bar) and combining the digital model of the upper shell and the digital model of the upper bar (and a digital model of the one or more upper projections, if not included with the bar).

The method may include using the software to digitally cut the intersecting areas of the lower portion and/or the projections of the lower portion. In implementations with multiple lower portions, implementations of the method may include digitally cutting the lower portion and/or projections of the lower portion configured to produce the maximum occlusal clearance first. The lower portion may be cut to provide an intersecting area which corresponds to the projection of the upper portion.

The method may then include exporting the lower portion digital model and the upper portion digital model to be produced. In various implementations, the method may include taking the model of the maximum occlusal clearance lower model and using the software to digitally cut more intersecting area and/or make smaller projections to provide a digital model of a lower portion with a decreased occlusal clearance. This model may then be exported to be produced via a 3D printing process. In various implementations, this process may repeat itself to produce any number of digital models of lower or upper portions. In a particular implementation, a single digital model of the upper portion may be exported and three different digital models of varying lower portions may be exported. In various implementations, after the digital models are exported, the corresponding lower portions and upper portions are 3D printed. In various implementations, they may be 3D printed using any material disclosed herein and using any 3D printer capable of handling those materials. As described above, various implementations of the method may include directly printing a sleep aid device from a digital impression without having to first create a mold of the sleep aid device which would be used to then print or produce the sleep aid device.

The sleep aid device described herein may be made of conventional materials used to make goods similar to these in the art, such as, by non-limiting example, plastic, monomers, metals, composites, and polymers. Those of ordinary skill in the art will readily be able to select appropriate materials and manufacture these products from the disclosures provided herein.

In places where the description above refers to particular implementations of sleep aid devices and implementing components, sub-components, methods and sub-methods, it should be readily apparent that a number of modifications may be made without departing from the spirit thereof and that these implementations, implementing components, sub-components, methods and sub-methods may be applied to other sleep aid devices.

What is claimed is:

1. A sleep aid device comprising;
an upper portion configured to receive a user's upper teeth, the upper portion comprising a first upper projection and a second upper projection;
a lower portion configured to receive a user's lower teeth coupled to the upper portion, the lower portion comprising a first lower projection and a second lower projection;
wherein a rear surface of the first lower projection is configured to contact a front surface of the first upper projection;
wherein a rear surface of the second lower projection is configured to contact a front surface of the second upper projection;
wherein the sleep aid device is configured to one of shift forward the user's mandible or increase the user's occlusal clearance.

2. The device of claim 1, wherein a bottom surface of the first upper projection and the second upper projection are configured to contact the lower portion.

3. The device of claim 1, wherein the sleep aid device is configured to shift forward the user's mandible and increase the user's occlusal clearance.

4. The device of claim 1, wherein the upper portion and the lower portion are configured to form an opening at the front of the device when the lower portion is coupled to the upper portion.

5. The device of claim 1, wherein the lower portion comprises a plurality of slots and the upper portion comprises a plurality of slots, each of the plurality of slots configured to receive a plurality of elastics.

6. The device of claim 1, wherein the device is configured to prevent a user's soft tissue from blocking an airway of the user.

7. A sleep aid device comprising;
an upper portion comprising:
an upper shell configured receive to a user's upper teeth;
an upper bar coupled below the upper shell; and
a first upper projection and a second upper projection coupled below the upper shell, wherein the first upper projection and the second upper projection extend from a rear portion of the upper portion; and
a lower portion comprising:
a lower shell configured to receive a user's lower teeth;
a lower bar coupled over the lower shell; and
a first lower projection and a second lower projection coupled over the lower shell;
wherein a rear surface of first lower projection is configured to contact a front surface of a first upper projection and a rear surface of the second upper projection is configured to contact a front surface of the second upper projection;
wherein the first lower projection and the second lower projection are configured to shift forward a user's mandible; and
wherein the first lower projection and the second lower projection are configured to provide occlusal clearance for a user.

8. The device of claim 7, wherein an entire bottom surface of the first upper projection and an entire bottom surface of the second upper projection are configured to contact the lower bar.

9. The device of claim 7, wherein an entire upper surface of the first lower projection and an entire upper surface of the second lower projection are configured to contact the upper bar.

10. The device of claim 7, wherein the upper shell and the lower shell are formed to match the user's teeth.

11. The device of claim 7, wherein the front surface of the first upper projection slopes towards the rear of the device from the upper shell.

12. The device of claim 7, wherein one of the upper bar and the lower bar are configured to provide occlusal clearance for the user.

13. The device of claim 7, wherein a height of the first lower projection is greater than a height of the first upper projection.

14. The device of claim 7, wherein the upper shell comprises a recess formed in a front sidewall of the upper shell.

15. The device of claim 7, wherein the lower shell comprises a recess formed in a front sidewall of the lower shell.

16. A sleep aid device kit comprising;
an upper portion configured to receive a user's upper teeth, the upper portion comprising a first upper projection and a second upper projection formed at a rear portion of the device;
a minimum lower portion configured to receive the user's lower teeth, the minimum lower portion comprising a first minimum projection and a second minimum projection;
a maximum lower portion configured to receive the user's lower teeth, the maximum lower portion comprising a first maximum projection and a second maximum projection;
wherein a rear surface of the first minimum projection and the first maximum projection are configured to contact a front surface of the first upper projection;
wherein a rear surface of the second minimum projection and the second minimum projection are configured to contact a front surface of the second upper projection; and
wherein one of a height of the projections of the maximum lower portion are greater than a height of the projections of the minimum lower portion or a length of the projections of the maximum lower portion are greater than a length of the projections of the minimum lower portion.

17. The kit of claim 16, further comprising a first intermediate lower portion comprising a first intermediate projection and a second intermediate projection, wherein a height of the first intermediate projection is greater than the height of the first minimum projection and less than the height of the first maximum projection.

18. The kit of claim 17, further comprising a second intermediate lower portion.

19. The kit of claim 16, wherein each lower portion is configured to shift a user's mandible forward and to increase a user's occlusal clearance.

20. The kit of claim 16, further comprising a second upper portion configured to receive a user's upper teeth.
